(19) United States
(12) Patent Application Publication
(10) Pub. No.: US 2025/0292600 A1
(43) Pub. Date: Sep. 18, 2025

(54) SYSTEMS AND METHODS FOR THE DETECTION AND CLASSIFICATION OF LIVE MICROORGANISMS USING THIN FILM TRANSISTOR (TFT) IMAGE SENSOR AND DEEP LEARNING

(71) Applicant: THE REGENTS OF THE UNIVERSITY OF CALIFORNIA, Oakland, CA (US)

(72) Inventors: Aydogan Ozcan, Los Angeles, CA (US); Yuzhu Li, Los Angeles, CA (US); Tairan Liu, Los Angeles, CA (US)

(73) Assignee: THE REGENTS OF THE UNIVERSITY OF CALIFORNIA, Oakland, CA (US)

(21) Appl. No.: 18/863,333

(22) PCT Filed: Apr. 25, 2023
(86) PCT No.: PCT/US2023/066216

§ 371 Date: Nov. 6, 2024

Related U.S. Application Data
(60) Provisional application No. 63/338,972, filed on May 6, 2022.

(51) Int. Cl. G06V 20/69; G06V 10/764; G06V 10/82

(52) U.S. Cl. CPC G06V 20/698; G06V 10/764; G06V 10/82; G06V 20/693

(57) ABSTRACT

A bacterial colony-forming-unit (CFU) detection system is disclosed that exploits a thin-film-transistor (TFT)-based image sensor array that saves ˜12 hours compared to the Environmental Protection Agency (EPA)-approved methods. A lensfree imaging modality was built using the TFT image sensor with a sample field-of-view of ˜10 cm². Time-lapse images of bacterial colonies cultured on chromogenic agar plates were automatically collected at 5-minute intervals. Two deep neural networks were used to detect and count the growing colonies and identify their species. When blindly tested with 265 colonies of E. coli and other coliform bacteria (i.e., Citrobacter and Klebsiella pneumoniae), the system reached an average CFU detection rate of 97.3% at 9 hours of incubation and an average recovery rate of 91.6% at ˜12 hours. This TFT-based sensor can be applied to various microbiological detection methods. The imaging field-of-view of this platform can be cost-effectively increased to >100 cm².

### RELATED APPLICATION

This application claims priority to U.S. Provisional Patent Application No. 63/338,972 filed on May 6, 2022, which is hereby incorporated by reference. Priority is claimed pursuant to 35 U.S.C. § 119 and any other applicable statute.

### TECHNICAL FIELD

The technical field generally relates to early screening and detection methods for the detection and/or identification of live microorganisms such as cells (prokaryotic or eukaryotic), viruses, fungi, bacteria, yeast, and multi-cellular organisms. More particularly, the technical field relates to systems and methods that periodically captures holographic microscopy images of bacterial growth on a growth plate and automatically analyzes these time-lapsed spatio-temporal patterns or holograms using multiple deep neural networks for the rapid detection and/or classification of the corresponding microorganism species.

### BACKGROUND

Bacterial infection has been a leading factor that causes millions of deaths each year in both developed and developing countries. The associated expenses of treating bacterial infections cost more than 4 billion dollars annually in the United States (US) alone. Therefore, the rapid and accurate detection of pathogenic bacteria is of great importance to human health in preventing such infectious diseases caused by e.g., contamination in food and drinking water. Among those pathogenic bacteria, Escherichia coli (E. coli) and other coliform bacteria are among the most common ones, and they indicate fecal contamination in food and water samples. The most basic and frequently used method of detecting E. coli and total coliform bacteria involves culturing the sample on a solid agar plate or liquid medium following the US Environmental Protection Agency (EPA)-approved protocols (e.g., EPA 1103.1 and EPA 1604 methods). However, these traditional culture-based methods usually take ≥24 hours for the final read-out and need visual recognition and counting of colony-forming units (CFUs) by microbiology experts. Although various nucleic acid-based molecular detection approaches have been developed for rapid bacteria detection with results ready in less than a few hours, they present lower sensitivity in general and have challenges to differentiate live and dead bacteria: in fact, there is no EPA-approved nucleic acid-based coliform sensing method that can be used for screening water samples.

Various other approaches have been developed to provide high sensitivity and specificity for the detection of bacteria based on different methods such as e.g., fluorimetry, solid-phase cytometry, fluorescence microscopy, Raman spectroscopy, and others: however, these systems, in general, do not work with large sample volumes (e.g., >0.1 L). As another alternative, Wang et al. demonstrated a complementary metal-oxide-semiconductor (CMOS) image sensor-based time-lapse imaging platform to perform early detection and classification of coliform bacteria. See Wang, H. et al., A. Early Detection and Classification of Live Bacteria Using Time-Lapse Coherent Imaging and Deep Learning. Light Sci. Appl. 2020, 9 (1), 118. https://doi.org/10.1038/s41377-020-00358-9. This method achieved more than 12 hours of detection time savings and provided species classification with >80% accuracy within 12-hours of incubation. The field-of-view (FOV) of the CMOS image sensor in this design was <0.3 cm², and therefore the mechanical scanning of the Petri dish area was required to obtain an image of the whole FOV of the cultured sample. Not only that this is time-consuming and requires additional sample scanning hardware, but it also brings some extra digital processing burden for image registration and stitching.

Recently, with the fast development of thin-film-transistors (TFT), the TFT technology has been widely used in the field of flexible display industry, radio frequency identification tags, ultrathin electronics, and large-scale sensors thanks to its high scalability, low-cost mass production (involving e.g., roll-to-roll manufacturing), low power consumption, and low heat generation properties. TFT technology has also been applied in the biosensing field to detect pathogens by transferring e.g., antibody-antigen binding, enzyme-substrate catalytic activity, or DNA hybridization into electrical signals. For example, a low-cost TFT nanoribbon sensor was developed by Hu et al. to detect the gene copies of E. coli and Klebsiella pneumoniae (K. pneumoniae) in a few minutes by using PH change due to DNA amplification. See Hu, C. et al., Ultra-Fast Electronic Detection of Antimicrobial Resistance Genes Using Isothermal Amplification and Thin Film Transistor Sensors, Biosens. Bioelectron. 2017, 96, 281-287. https://doi.org/10.1016/j.bios.2017.05.016. As another example, Salinas et al. implemented a ZnO TFT biosensor with recyclable plastic substrates for real-time E. coli detection. See Salinas, R. A. et al., Biosensors Based on Zinc Oxide Thin-Film Transistors Using Recyclable Plastic Substrates as an Alternative for Real-Time Pathogen Detection. Talanta 2022, 237, 122970. However, these TFT-based biosensing methods could not differentiate between live and dead bacteria and did not provide quantification of the CFU concentration of the sample under test.

### SUMMARY

Here, a TFT-based image sensor is used to build a real-time CFU detection system to automatically count the bacterial colonies and rapidly identify their species using deep learning. Because of the large FOV of the TFT image sensor (˜10 cm² or greater), there is no need for mechanical scanning of the agar plate, which enabled us to create a field-portable and cost-effective lensfree CFU detector as shown in FIGS. 2A-2C. This compact system includes sequentially switched red, green, and blue light-emitting diodes (LEDs) that periodically illuminate the cultured samples (E. coli, Citrobacter, and K. pneumoniae) as shown in FIG. 2C, and the spatio-temporal patterns of the samples are collected by the TFT image sensor, with an imaging period of 5 min. Two deep learning-based classifiers were trained to detect the bacterial colonies and then classify them into E. coli and total coliform bacteria. Blindly tested on a dataset populated with 265 colonies (85 E. coli CFU, 66 Citrobacter CFU, and 114 K. pneumoniae CFU), the TFT-based system was able to detect the presence of the colonies as early as ˜6 hours during the incubation period and achieved an average CFU detection rate of 97.3% at 9 hours of incubation, saving more than 12 hours compared to the EPA-approved culture-based CFU detection methods. For the classification of the detected bacterial colonies, an average recovery rate of 91.6% was achieved at ˜12 hours of incubation.

This TFT-based field-portable CFU detection system significantly benefits from the cost-effectiveness and ultra-large FOV of TFT image sensors, which can be further scaled up, achieving even lower costs with much larger FOVs based on e.g., roll-to-roll manufacturing methods commonly used in the flexible display industry. In some embodiments, the TFT image sensor(s) can be integrated with each agar plate to be tested, and can be disposed of after the determination of the CFU count, opening up various new opportunities for microbiology instrumentation in the laboratory and field settings.

In one embodiment, a system is disclosed for the detection and classification of live microorganism and/or colonies thereof in a sample using time-lapse imaging. The system includes a light source and a thin film transistor (TFT)-based image sensor located along an optical path originating from the light source. A growth plate containing growth medium thereon and containing the sample is interposed along the optical path and disposed adjacent to the TFT-based image sensor. A microcontroller or other circuitry in the system is configured to periodically illuminate the growth plate with light from the light source and capture time-lapse images of microorganisms and/or colonies thereof on the growth plate with the TFT-based image sensor. The system includes a computing device configured to execute image processing software to process and analyze time-lapse images of the microorganisms and/or colonies thereof on the growth plate and detect candidate microorganisms and/or colonies thereof in the time-lapse images.

In another embodiment, a method of detecting and classifying live microorganisms and/or colonies thereof using time-lapse imaging is disclosed. The method includes providing a growth plate containing an agar medium thereon and containing the sample: periodically illuminating the growth plate with at least one spectral band of illumination light from a light source: capturing time-lapse images of microorganisms and/or colonies thereof on the growth plate with the TFT-based image sensor; and detecting candidate microorganisms and/or colonies thereof in the time-lapse images with image processing software including a first trained deep neural network trained to detect true microorganisms and/or colonies thereof from non-microorganism objects and a second trained deep neural network that receives as an input at least one time-lapsed image or digitally processed time-lapsed image and outputs a species classification associated with the detected true microorganisms and/or colonies thereof.

### BRIEF DESCRIPTION OF THE DRAWINGS

FIG. 1 schematically illustrates a system for the early detection and classification of live microorganisms and/or colonies thereof in a sample using time-lapse imaging and deep learning.

FIGS. 2A-2C: illustrates a real-time CFU detection and classification system using a TFT image sensor. FIG. 2A: A photograph image of the lensfree imaging system, sample to be tested, and the laptop computer used for controlling the hardware. The chromogenic agar medium results in a gray-green color for E. coli colonies and a pinkish color for other coliform bacteria: furthermore, it inhibits the growth of different bacterial colonies or exhibits colorless colonies when other types of bacteria are present in the sample. FIG. 2B: a zoomed-in photograph of the TFT image sensor with a FOV of 32 mm×30 mm. FIG. 2C: a detailed illustration of the lensfree imaging modality. The red (620 nm), green (520 nm), and blue (460 nm) LEDs were switched on sequentially at 5-minute intervals to directly illuminate the cultured samples, which were imaged by the TFT image sensor in a single shot. The distance between the tri-color LED and the agar plate sample (z₁) is 15.5 cm, while the sample to sensor distance (z₂) distance is ˜5 mm.

FIG. 3: Illustrates a schematic of the workflow of the deep learning-based CFU detection and classification system. Eight (8) whole FOV RGB images are processed with 20-minute time intervals for the differential analysis to select the initial colony “candidates.” The digitally-cropped 8-frame RGB image sequence for each individual colony candidate is fed into the CFU detection neural network first. This neural network rejects various non-colony objects (among the initial colony candidates) such as dust and bubbles, achieving true colony detection. Next, the detected colonies are passed through the CFU classification neural network to identify their species (E. coli or other total coliforms, i.e., binary classification).

FIGS. 4A-4B: Visual evaluation of coliform bacterial colony early detection and classification using a TFT image sensor. FIG. 4A: whole FOV color images of E. coli at 11-hour incubation, Citrobacter at 13-hour incubation, and K. pneumoniae at 11-hour incubation. FIG. 4B: examples of the image sequence of each isolated colony growth. Three independent colony growth sequences were selected for each one of the bacteria species. The dashed line box labels the first colony detection time confirmed by the CFU detection neural network, and the dotted line box corresponds to the first classification time correctly predicted by the CFU classification neural network.

FIGS. 5A-5F: Quantitative performance evaluation of coliform colony early detection and classification using a TFT image sensor. FIGS. 5A, 5C, 5E: the colony detection rate as a function of the incubation time for E. coli, Citrobacter, and K. pneumoniae. The mean and standard deviation of the detection rate were calculated on 85 E. coli colonies, 66 Citrobacter colonies, and 114 K. pneumoniae colonies for each time point.

FIGS. 5B, 5D, 5F: the colony recovery rate as a function of the incubation time for E. coli, Citrobacter, and K. pneumoniae. The mean and standard deviation of the recovery rate were calculated on 85 E. coli colonies, 66 Citrobacter colonies, and 114 K. pneumoniae colonies for each time point.

FIG. 6: illustrates the bacterial colony candidate generation workflow (steps a-i). The image pre-processing steps a-i were performed on the acquired TFT images in order to select the colony candidates: the cropped videos of the colony candidates were then passed through a trained CFU detection neural network to determine the true positives and eliminate false positives.

FIG. 7 illustrates the network architectures for the CFU detection neural network and the CFU classification neural network. A Dense-Net design was adopted here, with the 2D convolutional layers replaced by the pseudo-3D convolutional blocks. The CFU detection and classification neural networks shared the same architecture, but the hyper-parameters [m, n, p, q] are selected to be different as indicated in FIG. 7.

### DETAILED DESCRIPTION OF ILLUSTRATED EMBODIMENTS

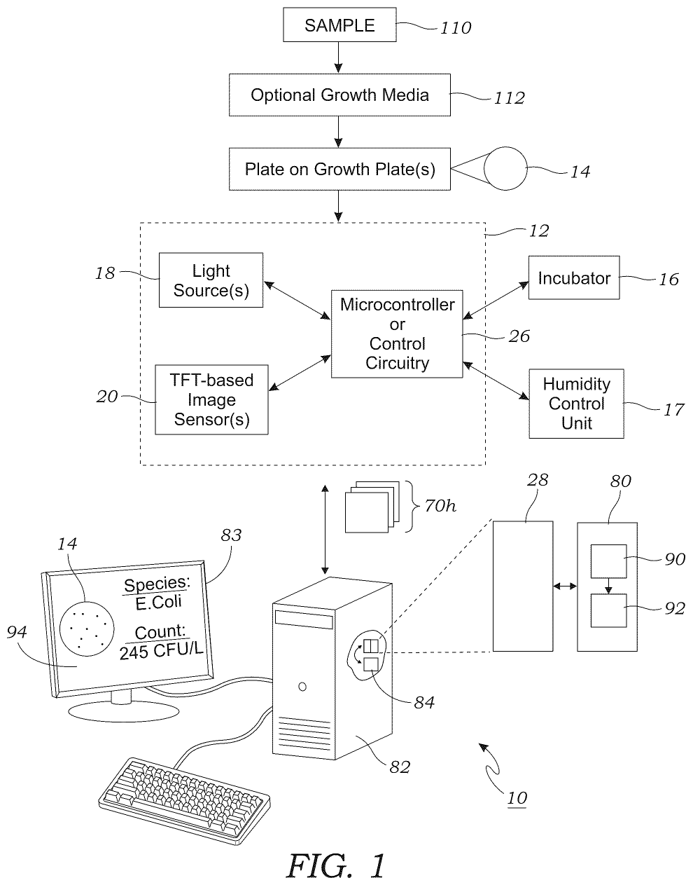

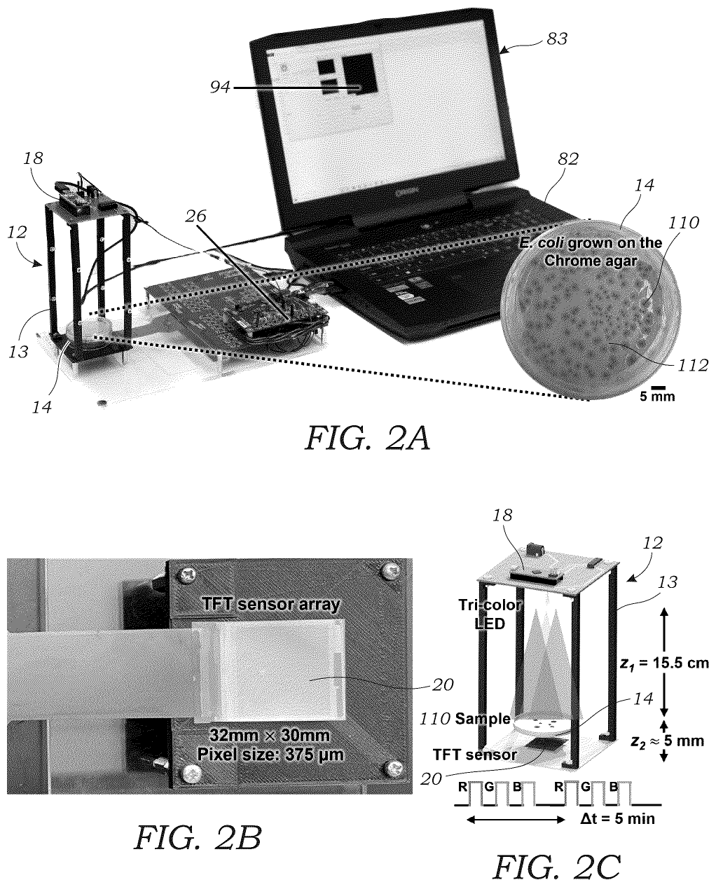

FIG. 1 illustrates a system 10 for the early detection and classification of live microorganisms and/or colonies thereof in a sample 110 using time-lapse imaging and deep learning according to one embodiment. Microorganisms include prokaryotic cells, eukaryotic cells (e.g., stem cells), fungi, bacteria, viruses, multi-cellular organisms (e.g., parasites) or clusters or films or colonies thereof. The system 10 includes a holographic imager device 12 (see also FIGS. 2A-2C) that is used to obtain time-lapsed images 70h of microorganism growth occurring on one or more growth plates 14 (e.g., Petri dish that contains chromogenic agar as a solid growth medium plus nutrients used to culture microorganisms or other growth medium(s) appropriate for the type of microorganism). The images 70h contain spatio-temporal patterns (e.g., holograms) of the sample 110.

The holographic imager device 12 includes a light source 18 that is used to direct light onto the sample 110. The light source 18 may include, as described herein, a tri-color LED module that sequentially switches red, green, and blue light-emitting diodes (LEDs). Other selectively actuated spectral bands may be used in the light source 18 in alternative embodiments. The holographic imager device 12 further includes a the TFT-based image sensor 20 that is disposed along an optical path of the light that is emitted from the light source 18. As seen in FIGS. 2A and 2C, the holographic imager device 12 includes a frame or housing 13 in which the light source 18 is located at one end (e.g., top) and the TFT-based image sensor 20 is located on an opposing end (e.g., bottom). The growth plate 14 that contains the sample 110 on the growth plate 14 is then interposed in the optical patch between the light source 18 and the TFT-based image sensor 20. In some embodiments, the growth plate 14 may be placed directly on the TFT-based image sensor 20. In other embodiments, the growth plate 14 may contain the TFT-based image sensor 20 directly on or within the growth plate 14. The TFT-based image sensor 20 may be reusable or, in some embodiments, disposable. An optional lens or set of lenses (not shown) may be located along the optical path and is/are used to magnify or de-magnify holograms captured with the TFT-based image sensor 20. The distance between the light source 18 and the sample 110 (i.e., the z₁ distance shown in FIG. 2C). is significantly greater (>>) than the distance between the sample 110 and the TFT sensor 20 (z₂). For example, in one embodiment, the z₁ distance ˜15.5 cm and the z₂ distance is ˜5 mm.

The holographic imager device 12 may include, in some embodiments, an incubator 16 to heat the one or more growth plates 14 and/or maintain the temperature at optimal setpoint temperature(s) or temperature range(s) for microorganism growth. A separate incubator 16 may also be used with the holographic imager device 12. The incubator 16 may include, in one embodiment, an optically transparent plate or substrate that contains heating elements therein that are used to adjust the temperature of the one or more growth plates 14. The incubator 16 may also include a fully or partially enclosed housing that accommodates the holographic imager device 12 along with the one or more growth plates 14. The holographic imager device 12 may also include one or more optional humidity control units 17 which are used to maintain the one or more growth plates 14 at a setpoint humidity level or range. The humidity control unit(s) 17 may be integrated with the incubator 16, the holographic imager device 12, or a separate component.

A series of time-lapsed images 70h of the microorganisms and/or colonies thereon on the growth plates 14 is used to identify microorganism colony candidates based on differential images obtained over time (i.e., time-lapsed images). The differential images (images 70h obtained at different times) include images of growing microorganisms and/or colonies but also includes non-microorganism objects such as dust, water bubbles or surface movement of the agar itself, and other artifacts. Image processing software 80 executed on a computing device 82 having one or more processors 84 is used to perform image pre-processing, differential analysis, colony mask segmentation, and candidate position localization, cropping of videos of colony candidates. However, some of these videos of colony candidates are not true microorganism colonies but may represent non-living objects or artifacts such as bubbles, dust, and the like which need to be masked or excluded. As explained herein, a first trained deep neural network (DNN) 90 is used by the image processing software 80 to detect the actual microorganisms and/or colonies and ignore the non-microorganism objects. Once the “true” microorganisms and/or colonies are selected, one or more of the time-lapsed image(s) and/or at least one digitally processed time-lapsed image (e.g., re-normalized images generated by division-based normalization as explained herein) are sent to a second trained deep neural network (DNN) 92 that is used to classify the species class or particular species of the microorganism(s) and/or colonies.

The system 10 is implemented with a holographic imager device 12 that includes a holographic imaging system that captures hologram images of growing microorganisms and/or colonies. A light source 18 (e.g., illumination module that includes tri-color light emitting diodes (LEDs)) illuminates the microorganisms and/or colonies thereof on the one or more growth plates 14 (which are incubated using the incubator 16) and holographic images 70h of the microorganisms and/or colonies thereof are captured with at least one TFT-based image sensor 20. The light source 18 preferably emits one or more illumination spectral bands that can be actuated (e.g., turned on/off) on demand. This may be accomplished through different spectral bands that are emitted by the light source 18 or through the use of filters that allow the passage of different spectral bands. The holographic imager device 12 may be placed inside a separate incubator 16 or the holographic imager device 12 may be integrated with the incubator 16. Notably, there is no need for scanning the one or more growth plates 14. A large filed-of-view (FOV) is captured by the TFT-based image sensor 20. In some embodiments, a lens or set of lenses is used to capture an even larger field of view of the one or more growth plates 14. Alternatively, a larger sized TFT-based image sensor 20 may be used. In one preferred embodiment, the captured FOV is at least 10 cm² or more. Even larger FOVs are contemplated including FOVs that are 100 cm² or more.

A microcontroller or control circuitry 26 is provided that is used to control the illumination of the light source 18, the incubator 16, the humidity control unit 17, and the capture of images with the TFT-based image sensor(s) 20. The microcontroller or control circuitry 26 may also communicate with the computing device 82, for example, to receive instructions and/or send data using a program 28 executed by the computing device 82. The microcontroller or control circuitry 26 may include one or more microprocessors, drivers, or the like located on a printed circuit board (PCB) that are used to operate various subsystems and transfer data. This includes the timing and sequence of illumination with the light source(s) 18, image acquisition from the TFT-based image sensor 20, etc. The microcontroller or control circuitry 26 may also be used to control the setpoint temperature or temperature range of the incubator 16. The control circuitry 26 may also be used to control the setpoint humidity level or humidity range of the incubator 16 using a humidity control unit 17. The microcontroller or control circuitry 26 may be located outside of the frame 13 as seen in FIG. 2A or, alternatively, it may be contained therein.

The system 10 includes at least one computing device 82 (e.g., personal computer, laptop, tablet PC, server, or the like) having one or more processors 84 therein which is used to execute image processing software 80 to process the images 70h obtained from the TFT-based image sensor(s) 20. The computing device 82 may be located with the holographic imager device 12 (e.g., a local implementation) or it may be remotely located therefrom (e.g., a remote computing device like a server). In other embodiments, multiple such computing devices 82 may be used (e.g., one to control the holographic imager device 12 and another to process the images 70h). In addition, the computing device 82 is, in some embodiments, able to control various aspects of the operation of the holographic imager device 12 using the microcontroller or control circuitry 26. For example, using a graphical user interface (GUI) 94 viewable on a display 83, the user can control aspects of the system 10 (e.g., periodicity or timing of image scans, TFT-based image sensor 20 operation, temperature control of incubator 16, transfer of image files 70h from TFT-based image sensor(s) 20 to computing device 82, etc.). The GUI 94 may also be used to display videos, classified colonies 102, colony counts, and display a colony growth map for viewing/interaction.

The computing device 82 executes image processing software 80 that includes the microorganism and/or colony detection deep neural network 90 which is used to identify the true microorganisms and/or colonies from other non-microorganism artifacts (e.g., dust, bubbles, speckle, etc.). The computing device 82 also executes a separate classification deep neural network 92 in the image processing software 80 that classifies the particular species class or actual species of microorganism and/or colonies. In an alternative embodiment, the functions of the first and second trained deep neural networks 90, 92 are combined into a single trained deep neural network (e.g., deep neural network 90). Multiple different species of microorganisms and/or colonies may be identified in a single sample. In one particular embodiment, the system 10 enables the rapid detection of Escherichia coli and total coliform bacteria (i.e., Klebsiella aerogenes and Klebsiella pneumoniae subsp. pneumoniae) in water samples. This automated and cost-effective live microorganism detection system 10 is transformative for a wide range of applications in microbiology by significantly reducing the detection time, also automating the identification of microorganisms and/or colonies, without labeling or the need for an expert.

To use the system 10, a sample 110 is obtained and optionally subject to a signal amplification operation where the sample is pre-incubated with growth media 112 (FIG. 1) for a period of time at elevated temperatures followed by filtration using, for example, a filter membrane. The sample 110 is a typically a fluid and may include, for example, a water sample (although it could be a food sample, a biological or other fluid sample). The filter membrane is then placed in physical contact with one or more growth plates 14 (e.g., agar surface of growth plate 14) for a period of time under light pressure to transfer the microorganisms (e.g., bacteria) to the agar growth medium in the growth plates 14 and then removed. However, in other embodiments, the sample 110 may also be placed directly on the growth plate 14 and spread using, for example, the L-shaped spreader disclosed herein. The one or more growth plates 14 are then covered placed in the holographic imager device 12 (e.g., upside down with the agar surface facing the TFT-based image sensor(s) 20) in/on the incubator 16. The growth plate 14 with the sample 110 is then allowed to incubate for several hours and is periodically imaged by the TFT-based image sensor(s) 20. In some embodiments, a single growth plate 14 is imaged by the TFT-based image sensor 20. In other embodiments, multiple growth plates 14 are imaged by the TFT-based image sensor 20. In the later embodiment, multiple TFT-based image sensors 20 may be used. In some embodiments, the TFT-based image sensor 20 is separate from the growth plate 14. In other embodiments, the growth plate 14 may be integrated with the TFT-based image sensor 20. This may be located on or within the growth plate 14.

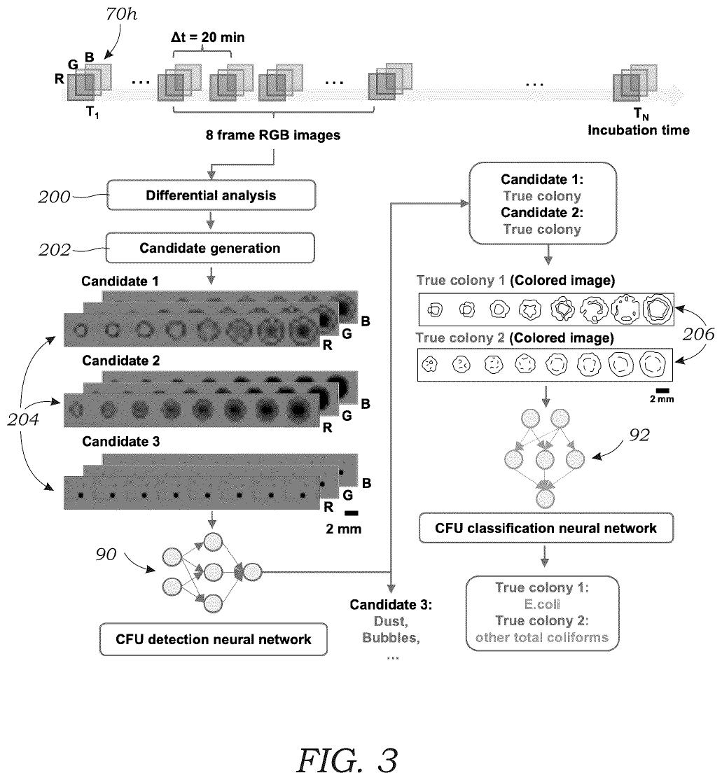

In one particular embodiment, a method of detecting and classifying live microorganisms and/or colonies thereof using time-lapse imaging includes loading the growth plate 14 containing a sample 110 into or onto an incubator 16. The one or more growth plates 14 are then illuminated with different spectral bands of light (e.g., colors) from the light source 18. Specifically, the growth plate 14 is periodically illuminated by different spectral bands of illumination light (e.g., color LEDs) in sequential fashion. Images 70h are captured by the TFT-based image sensor 20 at each color. Various periods between successive illumination may be used. In one embodiment, around five (5) minutes pass between illumination of the sample 110. This enables time-lapse images 70h of the growth plates 14 containing the microorganisms and/or colonies thereof to be taken using the holographic imager device 12. The time-lapse images 70h are then processed and true microorganisms and/or colonies are detected (and optionally counted) using the first trained deep neural network (DNN) 90 as seen in FIG. 3.

FIG. 3 illustrates the exemplary workflow of the deep learning-based CFU detection and classification system. Here, eight (8) whole FOV RGB images are processed with 20-minute time intervals for the differential analysis 200 to select the initial colony “candidates” for candidate generation 202. Of course, more or fewer images may be processed at different intervals. The digitally-cropped 8-frame RGB image sequence 204 (e.g., video) for each individual colony candidate (three such candidates are illustrated in FIG. 3) is fed into the CFU detection neural network 90 first. This neural network 90 rejects various non-colony objects (among the initial colony candidates) such as dust and bubbles (here candidate 3), achieving true colony detection (candidates 1 and 2). Next, the colored image sequences 206 of the true detected colonies are passed through the CFU classification neural network 92 to identify their species (e.g., E. coli or other total coliforms, i.e., binary classification). Finally, the detected microorganisms and/or colonies are then classified (and optionally counted) using the second trained deep neural network (DNN) 92.

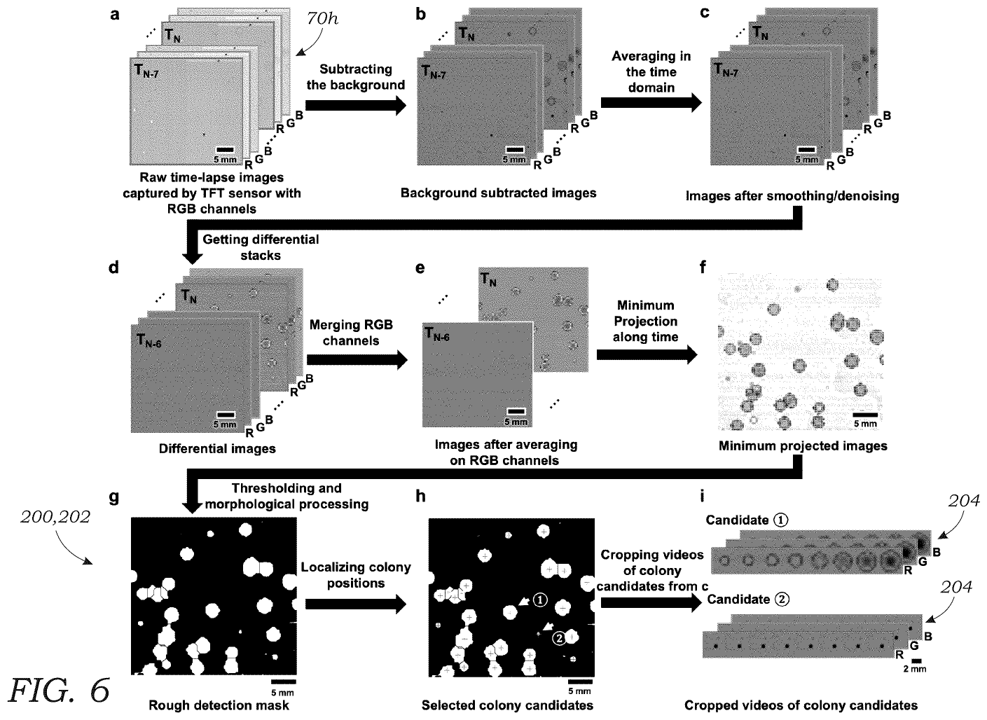

FIG. 6 illustrates further details on how differential analysis 200 is used to generate colony candidates 202 as illustrated in FIG. 3. In operation (a), raw time-lapse images are captured by the TFT sensor 20 with RGB channels. Background subtraction is performed to create background subtracted images as seen in operation (b). Next, the images are averaged in the time domain to smooth/denoise the images as seen in operation (c). In operation (d), differential stacks of the smoothed/denoised images obtained and the RGB channels are merged (averaged) as seen in operation (e). Next, in operation (f), minimum projection images are generated and subject the thresholding and morphological processing to generate a rough detection mask as seen in operation (g). Localized colony positions are identified colony candidate are selected as seen in operation (h). After colony candidates are selected, videos of the colony candidates in the RGB color channels are then cropped as RGB image sequences 204 as see in operation (i).

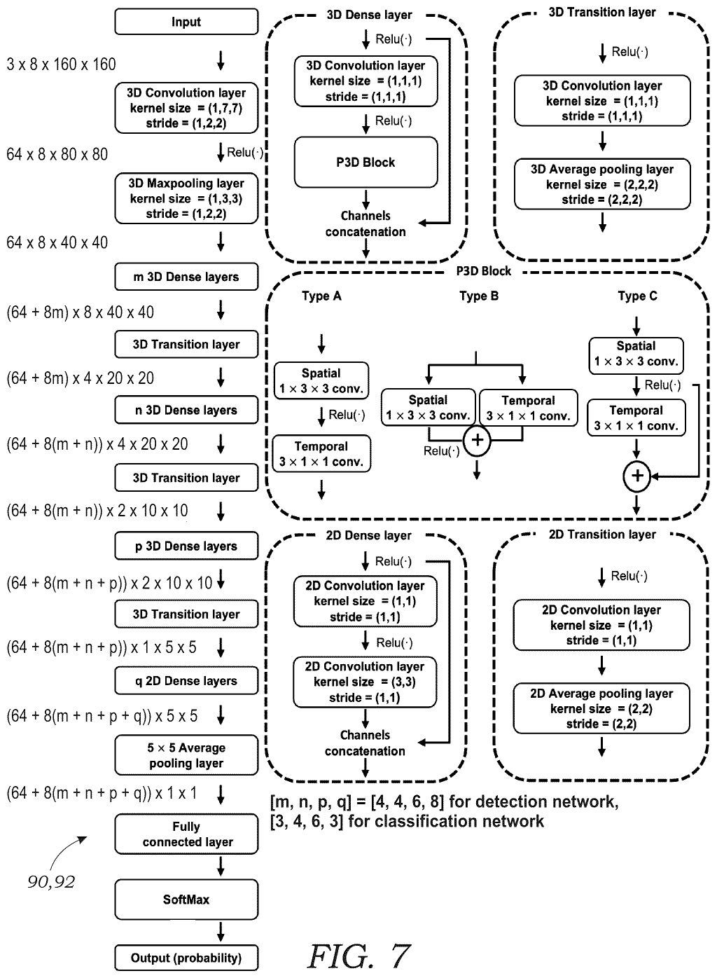

FIG. 7 illustrates the network architectures for the CFU detection neural network 90 and the CFU classification neural network 92. A Dense-Net design was adopted here, with the 2D convolutional layers replaced by the pseudo-3D convolutional blocks. The CFU detection and classification neural networks 90, 92 shared the same architecture, but the hyper-parameters [m, n, p, q] are selected to be different as indicated in FIG. 7.

### Experimental

### Results

The success of the system 10 was demonstrated by detecting and classifying the colonies E. coli and two other types of total coliform bacteria, i.e., Citrobacter and K. pneumoniae, on chromogenic agar plates, which result in a gray-green color for E. coli colonies and a pinkish color for other coliform bacteria, also inhibiting the growth of different bacterial colonies when other types of bacteria exist in the sample. Each sample 110 was prepared following the EPA-1103.1 method (see the Methods) using a Petri dish 14. After the sample 110 was prepared, it was directly placed on top of the TFT-based image sensor 20 as part of the lensfree imaging system 12, and the entire imaging modality (except the laptop 82 in FIG. 2A was placed inside an incubator 16 to record the growth of the colonies with 5-minute imaging intervals. For each time interval, three images 70h were collected sequentially using the TFT image sensor 20 under red (620 nm), green (520 nm), and blue (460 nm) illumination light. This multi-wavelength design allowed the monochromatic TFT image sensor 20 to reconstruct color images of the bacterial colonies and was mainly used to identify their species by exploiting the color information provided by the selective chromogenic agar medium 112. The recorded time-lapse images 70h were processed using the workflow shown in FIGS. 3 and 6, where a differential analysis 200 was used to generate the initial colony candidates 202, and two deep neural networks (DNNs) 90, 92 were trained to further screen the colony candidates to specifically detect the true colonies and infer their species class/species (see the Methods section). All these image processing steps take <25 sec using an Intel Core i7-7700 CPU-powered computer, consuming <1 GB of memory (without the need for GPUS).

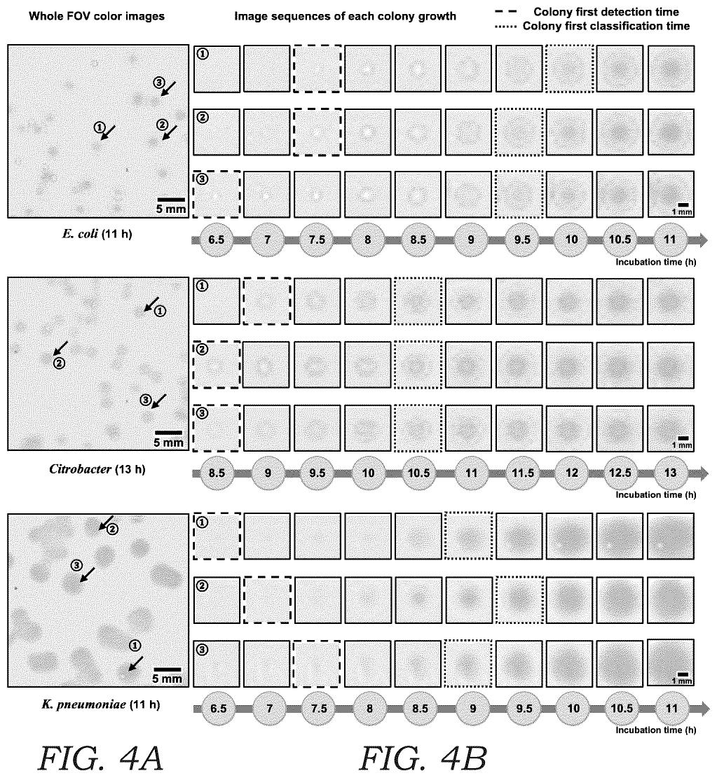

The presented TFT imaging system 10 periodically captures the images 70h of the agar plate 14 under test based on lensfree in-line holography: however, due to its large pixel size (375 μm) and relatively small sample to sensor distance (˜5 mm, which is equal to the thickness of the agar), a free space backpropagation step is not needed. By directly using the raw intensity images 70h as part of the RGB color channels and calibrating the background, the color images of the agar plate can be generated in <0.25 sec after the TFT images are recorded. FIGS. 4A-4B shows examples of images (in color) of E. coli, Citrobacter, and K. pneumoniae colonies at different stages of their growth, captured by the system 10. Consistent with the EPA-approved method (EPA-1103.1), E. coli colonies exhibit gray-green colors, while Citrobacter and K. pneumoniae colonies exhibit pinkish color using the chromogenic agar.

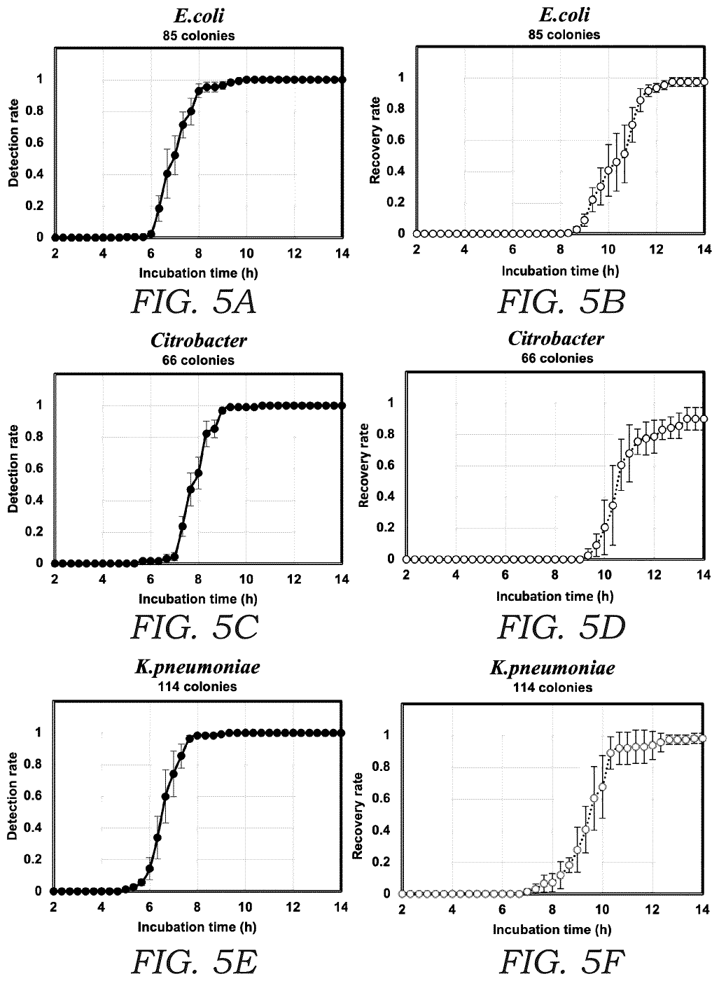

Based on the imaging performance of the TFT-based CFU detection system 10 summarized in FIGS. 4A-4B, its early detection and classification performance was quantified as shown in FIGS. 5A-5F. For this, the detection and the classification neural network models was trained (see the Methods for training details) on a dataset of 442 colonies (128 E. coli colonies, 126 Citrobacter, and 188 K. pneumoniae colonies) captured from 17 independent experiments. The testing dataset was populated using 265 colonies from 13 independent experiments, which had a total of 85 E. coli colonies, 66 Citrobacter colonies, and 114 K. pneumoniae colonies. The detection rate was defined as the ratio of the number of true colonies confirmed by the CFU detection neural network 90 out of the total colony number counted by an expert after 24-hour incubation. FIGS. 5A, 5C, 5E shows the detection rate achieved in the blind testing phase as a function of the incubation time. As shown in FIGS. 5A, 5C, 5E, >90% detection rate was achieved at 8 hours of incubation for E. coli, 9 hours for Citrobacter, and 7 hours 40 minutes for K. pneumoniae. Furthermore, a 100% detection rate was obtained within 10 hours of incubation for E. coli, 11 hours for Citrobacter, and 9 hours 20 minutes for K. pneumoniae. Compared to the EPA-approved standard read-out time (24 hours), the TFT-based CFU detection system 10 achieved >12 hours of time-saving. Moreover, from the detection rate curves reported in FIGS. 5A-5F, one can also qualitatively infer that the colony growth speed of K. pneumoniae is larger than E. coli which is larger than Citrobacter because the earliest detection times for E. coli, Citrobacter, and K. pneumoniae colonies were 6 hours, ˜6.5 hours and ˜5.5 hours of incubation, respectively.

To quantify the performance of the bacterial colony classification neural network 92, the recovery rate was defined as the ratio of the number of correctly classified colonies to the total number of colonies counted by an expert after 24-hour incubation. FIGS. 5B, 5D, 5F show the recovery rate curves over all the blind testing experiments as a function of the incubation time. One can see that a recovery rate of >85% was achieved at 11 hours 20 minutes for E. coli, at 13 hours for Citrobacter, and at 10 hours 20 minutes for K. pneumoniae. It is hard to achieve a 100% recovery rate for all the colonies since some of the late growing “wake-up” colonies could not grow to a sufficiently large size with the correct color information even after 24 hours of incubation. FIGS. 5A-5F also reveals that there exists approximately a 3-hour time delay between the colony detection time and species identification time: this time delay is expected since more time is needed for the detected colonies to grow larger and provide discernable color information for the correct classification of their species.

### DISCUSSION

Note that the presented results in FIGS. 5A-5F represent a conservative performance of the TFT-based CFU detection method since the ground truth colony information was obtained after 24 hours of incubation. In the early stages of the incubation period, some bacterial colonies did not even exist physically. Therefore, if the existing colony numbers for each time point were used as the ground truth, even higher detection and recovery rates could be reported in FIGS. 5A-5F.

Overall, the performance of the TFT-based CFU detection system 10 is similar to the CMOS-based time-lapse hologram imaging method in terms of the colony detection speed. However, due to its large pixel size (375 μm) and limited spatial resolution, the TFT-based method has a slightly delayed colony classification time. With its ultra-large imaging FOV (˜10 cm²), the TFT-based CFU detection method eliminates (1) the time-consuming mechanical scanning of the Petri dish and the related optomechanical hardware, and (2) the image processing steps for image registration and stitching that would both be required due to the limited FOV of CMOS-based imagers. In addition to saving image processing time, this also helps the system to increase the CFU detection sensitivity as the system 10 is free from any image registration and stitching artifacts and therefore, it can precisely capture minute spatio-temporal changes in the agar caused by bacterial colony growth at an early stage. Due to the massive scalability of the TFT-based image sensor 20 arrays, the imaging FOV of the platform can be further increased to several tens to hundreds of cm² in a cost-effective manner, which could provide unprecedented levels of imaging throughput for automated CFU detection using e.g., roll-to-roll manufacturing of TFTs, as employed in the flexible display industry.

Another prominent advantage of the TFT-imager based detection system 10 is that it can be adapted to image a wide range of biological samples 110 using cost-effective and field-portable interfaces. Should the users have any contamination concerns, the TFT image sensor 20 shown in FIGS. 2B, 2C can be replaced and even used in a disposable manner (e.g., integrated as part of the growth plate 14 (e.g., Petri dish)). Furthermore, the heat generated by the TFT image sensor 20 during the data acquisition process is negligible, ensuring that the biological samples 110 can grow at their desired temperature without being perturbed. Finally, the TFT-based CFU detection system 10 is user-friendly and easy-to-use because there is no need for complex optical alignment, high precision mechanical scanning stages, or image registration/alignment steps.

The presented CFU detection system 10 using TFT image sensor 20 arrays provides a high-throughput, cost-effective, and easy-to-use solution to perform early detection and classification of bacterial colonies, opening up unique opportunities for microbiology instrumentation in the laboratory and field settings.

### Materials and Methods

### Sample Preparation

All the bacterial sample preparations were performed at a Biosafety Level 2 laboratory in accordance with the environmental, health, and safety rules of the University of California, Los Angeles. E. coli (Migula) Castellani and Chalmers (ATCCR) 25922™), Citrobacter (ATCCR: 43864™), and K. pneumoniae subsp. pneumoniae (Schroeter) Trevisan (ATCCR: 13883™) were used as the culture microorganisms. CHROMagar™ ECC (product no. EF322, DRG International, Inc., Springfield, NJ, USA) chromogenic substrate mixture was used as the solid growth medium to detect E. coli and other total coliform colonies.

For each time-lapse imaging experiment, a bacterial suspension in a phosphate-buffered solution (PBS) (product no. 20-012-027, Fisher Scientific, Hampton, NH, USA) was prepared from a solid agar plate incubated for 24 hours. The concentration of the suspension was measured using a spectrophotometer (model no. ND-ONE-W, Thermo Fisher). Then, a serial dilution was performed in PBS to finally reach a concentration of ˜10³ CFUs/mL. Around 100 μL diluted suspension with ˜100 CFUs was spread on a CHROMagar™ ECC plate using an L-shaped spreader (product no. 14-665-230, Fisher Scientific, Hampton, NH, USA). Next, the growth plate 14 was covered with its lid, inverted, and placed on the TFT image sensor 20, which was placed with the whole imaging system 12 into an incubator 16 (product no. 151030513, ThermoFisher Scientific, Waltham, MA, USA) kept at 37±0.2° C.

Additionally, CHROMagar™ ECC plates were prepared ahead of time using the following method. CHROMagar™ ECC (6.56 g) was mixed with 200 mL of reagent grade water (product no. 23-249-581, Fisher Scientific, Hampton, NH, USA). The mixture was then heated to 100° C. on a hot plate while being stirred regularly using a magnetic stirrer bar. After cooling the mixture to ˜50° C., 10 mL of the mixture was dispensed into each Petri dish (60 mm×15 mm) (product no. FB0875713A, Fisher Scientific, Hampton, NH, USA). When the agar plates solidified, they were sealed using parafilm (product no. 13-374-16, Fisher Scientific, Hampton, NH, USA), and covered with aluminum foil to keep them in the dark before use. These plates were stored at 4° C. and were used within two weeks after preparation.

### Imaging Setup

The field-portable CFU imager 12 includes an illumination module that contains the light source(s) 18 and a TFT-based image sensor 20. The light from a tri-color LED light source 18 directly illuminates the samples 110 and forms in-line holograms on the TFT image sensor 20 (JDI, Japan Display Inc., Japan). The TFT module includes a controlling printed circuit board (PCB) that provides the illumination and image capture control signal and an image sensor 20 (with 80×84 pixels, pixel size=375 μm). For the illumination module, a tri-color LED (EDGELEC) was controlled by a microcontroller 26 (Arduino Micro, Arduino LLC) through a constant current LED driver (TLC5916, Texas Instrument, TX, USA) to sequentially provide the red (620 nm), green (520 nm), and blue (420 nm) illumination beams. The microcontroller 26, the LED driver, and the tri-color LED were all integrated on a single PCB, which was powered by a 5V-1A voltage adapter and communicated with the TFT PCB through the LED power signal.

The illumination light passes through the transparent solid agar and forms the lensfree images of the growing bacterial colonies on the TFT image sensor 20. The distance between the LED and the sample (i.e., the z₁ distance shown in FIG. 2C), is ˜15.5 cm, which is large enough to make the illumination light uniformly cover the whole sample surface. The distance between the sample 110 and the TFT sensor 20 (22) is roughly equal to the thickness of the solid agar, which is ˜5 mm. The mechanical support material for the PCB, the sample, and the sensor were custom fabricated using a 3D printer (Objet30 Pro, Stratasys, Minnesota, USA).

### Image Data Acquisition

Time-lapse imaging experiments were conducted to collect the data for both the training and testing phases. The CFU imaging modality captured the time-lapse images 70h of the agar plate under test every 5 min under red, green, and blue illuminations. A controlling program 28 with a graphical user interface (GUI) 94 was developed to perform the illumination switching and image capture automatically. The raw TFT hologram images 70h were saved in 12-bit format. After the experiments were completed, the samples were disposed of as solid biohazardous waste. In total, the time-lapse TFT hologram images 70h of 889 E. coli colonies from 17 independent experiments were collected to initially train the CFU detection neural network model. In addition to this, 442 bacterial colonies (128 E. coli, 126 Citrobacter, and 188 K. pneumoniae) were populated from 17 new agar plates and used to train (1) the final CFU detection neural network 90 (through transfer learning from the initial detection model) and (2) the CFU classification neural network 92. A third independent dataset of 265 colonies from 13 new experiments was used to test the trained neural network models blindly.

### Bacterial Colony Candidate Selection

The entire candidate selection workflow consists of image pre-processing, differential analysis, colony mask segmentation, and candidate position localization, following the operations illustrated in FIG. 6 (operations a-i). For each time point, three raw TFT images 70h (red, green, and blue channels) were obtained over a FOV of ˜10 cm². After getting the TFT images I_{N_raw, C}, where N refers to the N-th image obtained at T_{N} and C represents the color channels, R (red), G (green), and B (blue), a series of pre-processing operations were performed to enhance the image contrast. First, as shown in operations a-b of FIG. 6, the images were 5 times interpolated and normalized by directly subtracting the first frame at T₀. After this normalization step, the background regions had ˜0 signal, while the regions representing the growing colonies had negative values because the colonies partially blocked and scattered the illumination light. Then, by adding 127 and saving the images as unsigned 8-bit integer arrays, the current frame at T_{N} was scaled to 0-127, noted as I_{N_norm, C}. Following the operations b-c in FIG. 6, I_{N_norm, C} was averaged as shown in Equation (1) to perform smoothing in the time domain, which yields I_{N_denoised, C}:

$${I}_{{N}_{d e n o i s e d} , C} = \frac{1}{3} {\sum }_{n = N - 2}^{n = N} {I}_{{n}_{n o r m} , C} \qquad ( 1 )$$

To further improve the sensitivity of the system, differential images I_{N_diff} averaged on three color channels were calculated as follows:

$${I}_{N \_ diff} = \frac{1}{3} {\sum }_{C = R , G , B} ( {I}_{N \_ denoised , C} - {I}_{( N - 1 ) \_ denoised , C} ) \qquad ( 2 )$$

By this operation, the signals of static artifacts were suppressed, and the spatio-temporal signals of the growing colonies were enhanced as ring-shaped patterns. Next, a pixel-wise minimum intensity projection was performed, as shown in operations e-f of FIG. 6, to project the minimum intensity of the differential images from I_{(N-7)_diff} to I_{N_diff}, yielding the image I_{N_projection}. Following this step, with an empirically set intensity threshold, I_{N_projection} was segmented into a binary mask. After morphological operations to fill the ring-shaped patterns and a watershed-based division of clustered regions, M_{N} was obtained as presented in operation g of FIG. 6. Based on this binary mask, M_{N}, the connected components were extracted and localized their centroids as shown in operation h of FIG. 6. These centroid coordinates were dynamically updated for each time point to ensure maintaining the localization at the center of the growing colonies.

Despite this pre-processing of the acquired TFT images 70h, there are still some time-varying non-colony objects that can be selected as false colony candidates (such as bubbles, dust, or other features created by the uncontrolled motion of the agar surface). Therefore, a deep neural network 90 was trained to further screen each colony candidate to eliminate false positives, the details of which will be discussed in the next subsection.

### DNN-Based Detection of Bacterial Colony Growth

The time-lapse video 204 of each colony candidate region across 8 frames of I_{N_denoised, C} was cropped as shown in operation i of FIG. 6. These videos 204 were then up-sampled in the spatial domain and organized as a four-dimensional array (3×8×160×160, i.e., color channels×number of frames×x×y) to be fed into the CFU detection neural network 90, which adopted the architecture of Dense-Net, but with 2D convolutional layers replaced by pseudo-3D convolutional layers (see FIG. 7). The weights of this CFU detection DNN 90 were initialized with a pre-trained model obtained on the E. coli CFU dataset with a single illumination wavelength of 515 nm. This pre-trained model was obtained using a total of 889 colonies (positives) and 159 non-colony objects (negatives) from 17 independent agar plates. Then, this initial neural network model was transferred to the multiple-species image dataset with multi-wavelength illumination, using 442 new colonies and 135 non-colony objects from another 17 independent agar plates. Both the positive image dataset and the negative image dataset were augmented across the time domain with different starting and ending time points, resulting in more than 10,000 videos used for training. A 5-fold cross-validation strategy was adopted to select the best hyper-parameter combinations. Once the hyper-parameters were decided, all the collected data were used for training to finalize the CFU detection neural network 90. Data augmentation, such as flipping and rotation, was also applied when loading the training dataset.

The network model 90 was optimized using the Adam optimizer with a momentum coefficient of (0.9, 0.999). The learning rate started as 1×10⁻⁴ and a scheduler was used to decrease the learning rate with a coefficient of 0.8 at every 10 epochs. The batch size was set to 8. The loss function was selected as:

$$l ( p , g ) = {\sum }_{k = 1}^{k = K} {\sum }_{l = 1}^{2} - {w}_{l} \cdot {g}_{k , l} \cdot log ( \frac{exp ( {p}_{k , l} )}{exp ( {p}_{k , 1} ) + exp ( {p}_{k , 2} )} ) \qquad ( 3 )$$

where p is the network output, which is the probability of each class before the SoftMax layer, g is the ground-truth label (which is equal to 0 or 1 for binary classification), K is the total number of training samples in one batch, w is the weight assigned to each class, defined as w=1−d where d is the percentage of the samples in one class. The training process was performed using a GPU (GTX1080Ti) which took ˜5 hours to converge. With a decision threshold of 0.5, the CFU detection neural network 90 converged with 92.6% sensitivity and 95.8% specificity. In the testing phase, the decision threshold was set to be 0.99, which achieved 100% specificity.
   DNN-Based Classification of E. coli and Other Total Coliform Colonies

To classify the species of the detected bacterial colonies, a second DNN-based classifier 92 was built. The CFU classification neural network 92 was trained on the same multi-wavelength dataset populated with 442 colonies (128 E. coli colonies, 126 Citrobacter colonies, and 188 K. pneumonia colonies). The input of the classification DNN 92 was organized into a four-dimensional array (3×8×160)×160, i.e., color channels×number of frames×x×y), but with a different normalization method. Different from the background subtraction normalization adopted for the CFU detection neural network 92, for the classification DNN 92, the network input was re-normalized by dividing the background intensities obtained at the first time point T₀. This division-based normalization was performed on three color channels so that the background would be normalized to ˜1 in the three channels, revealing a white color in the background. Through this operation, the color variations across different experiments were minimized, improving the generalization capability of the classification DNN 92.

The network structure of the classification DNN 92 was the same as the CFU detection network 90 but with some differences in the hyper-parameter selection (see FIG. 7). The classification neural network model was initialized randomly and optimized using the Adam optimizer with a momentum coefficient of (0.9, 0.999). The learning rate started with 1×10⁻³ and a scheduler was used to decrease the learning rate with a coefficient of 0.7 at every 30 epochs. The batch size was also set to 8. The classification neural network also used the weighted cross-entropy loss function as shown in Equation (3). The training process was performed using a GPU (GTX1080Ti) which took ˜5 hours to converge. A decision threshold of 0.5 was used to classify the E. coli colonies and other total coliform colonies in the training process, achieving 91% and 97% accuracy, respectively. In the testing phase, the decision threshold was set to be 0.8, which achieved 100% classification accuracy. In addition, a colony size threshold of 4.5 mm² was used in the testing phase to ensure that only colonies that are large enough to identify their species were passed through the classification network 92.

While embodiments of the present invention have been shown and described, various modifications may be made without departing from the scope of the present invention. For example, multiple TFT-based image sensors may be used to perform detection and classification over larger areas or different growth plates. The invention, therefore, should not be limited, except to the following claims, and their equivalents.

1. A system for the detection and classification of live microorganism and/or colonies thereof in a sample using time-lapse imaging comprising:
a light source;
a thin film transistor (TFT)-based image sensor located along an optical path originating from the light source;
a growth plate containing growth medium thereon and containing the sample interposed along the optical path and disposed adjacent to the TFT-based image sensor;
a microcontroller or other circuitry configured to periodically illuminate the growth plate with light from the light source and capture time-lapse images of microorganisms and/or colonies thereof on the growth plate with the TFT-based image sensor; and
a computing device configured to execute image processing software to process and analyze time-lapse images of the microorganisms and/or colonies thereof on the growth plate and detect candidate microorganisms and/or colonies thereof in the time-lapse images.

2. The system of claim 1, further comprising an incubator integrated with the light source, TFT-based image sensor, and growth plate.

3. The system of claim 1, wherein the light source comprises one or more selectively actuated spectral bands.

4. The system of claim 1, wherein the image processing software is configured to receive the captured time-lapse images of the microorganisms and/or colonies thereof on the growth plate, the image processing software configured to: (1) detect candidate microorganisms and/or colonies thereof in the time-lapse images using a first trained deep neural network trained to detect true microorganisms and/or colonies thereof from non-microorganism objects, and (2) output a species class associated with the detected true microorganisms and/or colonies thereof using a second trained deep neural network that receives as an input at least one time-lapsed image or at least one digitally processed time-lapsed image of the true microorganisms and/or colonies thereof.

5. The system of claim 1, wherein the microorganisms comprise a prokaryotic cell, a eukaryotic cell, bacteria, fungi, virus, multi-cellular organism, or clusters, films, or colonies thereof.

6. The system of claim 1, wherein the computing device comprises a local and/or remote computing device(s).

7. The system of claim 1, wherein a lens or set of lenses are used to magnify or de-magnify holograms of the microorganisms and/or colonies thereof onto the TFT-based image sensor.

8. The system of claim 1, wherein the TFT-based image sensor captures a field-of-view of at least 10 cm².

9. The system of claim 1, wherein the TFT-based sensor is integrated on or within the growth plate.

10. The system of claim 1, wherein the TFT-based sensor is disposable.

11. The system of claim 1, wherein the growth medium comprises chromogenic agar plates.

12. A method of using the system of claim 1, comprising:
placing the growth plate comprising the sample within the optical path;
periodically illuminating the growth plate with the light source, wherein the periodic illumination comprises sequentially illuminating the growth plate at one or more spectral bands of illumination; and
obtaining a plurality of time-lapsed images of microorganisms and/or colonies thereof on the growth plate.

13. The method of claim 12, further comprising processing the time-lapsed images of the microorganisms and/or colonies thereof on the growth plate with image processing software, the image processing software further configured to detect candidate microorganisms and/or colonies thereof in the time-lapse images based on differential image analysis in the time-lapse holographic images and further including a first trained deep neural network trained to detect true microorganisms and/or colonies thereof from non-microorganism objects and a second trained deep neural network that receives as an input at least one time-lapsed image or at least one digitally processed time-lapsed image of the true microorganisms and/or colonies thereof and outputs a species class associated with the detected true microorganisms and/or colonies thereof.

14. The method of claim 13, wherein the microorganisms comprise a prokaryotic cell, a eukaryotic cell, bacteria, fungi, virus, multi-cellular organism, or clusters, films, or colonies thereof.

15. The method of claim 12, wherein the sample comprises one or more of a water sample, a food sample, a biological or other fluid sample.

16. A method of detecting and classifying live microorganisms and/or colonies thereof using time-lapse imaging comprising:
providing a growth plate containing a growth medium thereon and containing the sample;
periodically illuminating the growth plate with at least one spectral band of illumination light from a light source;
capturing time-lapse images of microorganisms and/or colonies thereof on the growth plate with the TFT-based image sensor; and
detecting candidate microorganisms and/or colonies thereof in the time-lapse images with image processing software including a first trained deep neural network trained to detect true microorganisms and/or colonies thereof from non-microorganism objects and a second trained deep neural network that receives as an input at least one time-lapsed image or digitally processed time-lapsed image and outputs a species classification associated with the detected true microorganisms and/or colonies thereof.

17. The method of claim 16, wherein the microorganisms comprise a prokaryotic cell, a eukaryotic cell, bacteria, fungi, virus, multi-cellular organism, or clusters, films, or colonies thereof.

18. The method of claim 16, wherein the time-lapsed images are obtained several times each hour over several hours.

19. The method of claim 16, wherein the TFT-based image sensor captures magnified or de-magnified holograms of the microorganism objects and/or microorganism colonies thereof using a lens or set of lenses.
